(19) United States
(12) Patent Application Publication
(10) Pub. No.: US 2022/0193295 A1
(43) Pub. Date: Jun. 23, 2022

(54) Inflatable and Scented Flower And Vase

(71) Applicant: Fredrick Smith, Cuthbert, GA (US)

(72) Inventor: Fredrick Smith, Cuthbert, GA (US)

(21) Appl. No.: 17/557,631

(22) Filed: Dec. 21, 2021

Related U.S. Application Data
(60) Provisional application No. 63/128,255, filed on Dec. 21, 2020.

(51) Int. Cl. A61L 9/01; A61L 9/12

(52) U.S. Cl. CPC A61L 9/01; A61L 2209/11; A61L 9/12

(57) ABSTRACT

An inflatable and scented vase and flower bouquet includes a housing having an outer surface configured in the form of a vase having flowers extending upwardly from a top of the vase, the housing having a singular construction and defining a single interior space that may be inflated to have the appearance of an authentic vase and flower bouquet. The inflatable bouquet may include a scent dispenser operative to dispense a fragrance associated with a flower depicted by the flower portion of the housing. A weight may be situated in the interior space to prevent the housing from floating away if inflated with helium.

### REFERENCE TO RELATED APPLICATIONS

This is a non-provisional application claiming the priority of provisional application 63/128,255 filed on Dec. 21, 2020 titled Inflatable and Scented Flower and Vase.

### BACKGROUND OF THE INVENTION

This invention relates generally to flower and vase combinations and, more particularly, to a vase and flower bouquet that is artificial and inflatable while generating a scent indicative of a real flower or flower arrangement.

A simple flower arrangement or bouquet of multiple stems and flowers placed attractively in a vase containing a suitable amount of water are known and are popularly purchased and given to persons who are fond of one another, such as spouses, girlfriends, or significant others. It is also popular to purchase and give flowers on special occasions such as anniversaries, birthdays, or to celebrate a special event such a winning a contest, participating in recital, or just to show appreciation. It is also common to attach inflated balloons to the vase of flowers.

Although presumably effective for their intended purposes, the prior art devices and products have the disadvantages that live flowers shed their leaves and petals, adding traditional balloons can dd significantly to the cost, and often require the inconvenience of having to plan ahead, e.g., calling a flower shop, arranging for delivery, and otherwise removing the possibility of a truly spontaneous “just because” reason for making an otherwise positive gesture.

Therefore, it would be desirable to have vase and flower combination that is artificial and inflatable yet has a realistic configuration and gives off a pleasant smell associated with the type of flower that is depicted in the inflatable bouquet. Further, it would be desirable to have vase and flower combination thar is generally flat and easy to store when deflated (a storage configuration) and expands to a full-size vase with flowers that is full-size when inflated (i.e., a deployed configuration. In addition, it would be desirable to have vase and flower combination that includes a weight so that the invention will not float away inadvertently.

### SUMMARY OF THE INVENTION

An inflatable and scented vase and flower bouquet includes a housing having an outer surface configured in the form of a vase having flowers extending upwardly from a top of the vase, the housing having a singular construction and defining a single interior space that may be inflated to have the appearance of an authentic vase and flower bouquet. The inflatable bouquet may include a scent dispenser operative to dispense a fragrance associated with a flower depicted by the flower portion of the housing. A weight may be situated in the interior space to prevent the housing from floating away if inflated with helium.

Therefore, a general object of this invention is to provide a vase and flower combination that is inflatable upon an introduction of air (ambient air or with helium).

Another object of this invention is to provide an inflatable flower and vase combination, as aforesaid, having a sent dispenser that emits a predetermined scent associated with a flower depicted by the inflatable device.

Yet another object of this invention is to provide an inflatable flower and vase combination, as aforesaid, that is easy to store in a retail store, is inexpensive, and is able to be an impulse purchase such as in a supermarket or other retailers of flowers and gift balloons.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1A is a perspective view of an inflatable scented flower and vase device according to a preferred embodiment of the present invention, illustrated in a deflated configuration;

FIG. 1B is a perspective view of the inflatable scented flower and vase device as in FIG. 1A, illustrated in an inflated configuration;

FIG. 2 is a side view of the inflatable scented flower and vase device as in FIG. 1B, illustrated on an enlarged scale;

FIG. 3A is a sectional view taken along line 3A-3A of FIG. 2; and

FIG. 3B is a block diagram illustrating the electric or electronic components of the present invention.

### DESCRIPTION OF THE PREFERRED EMBODIMENT

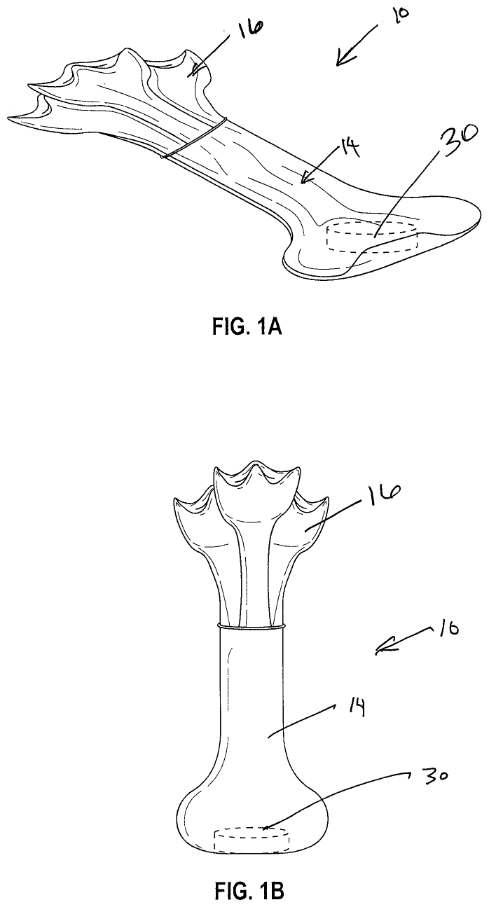

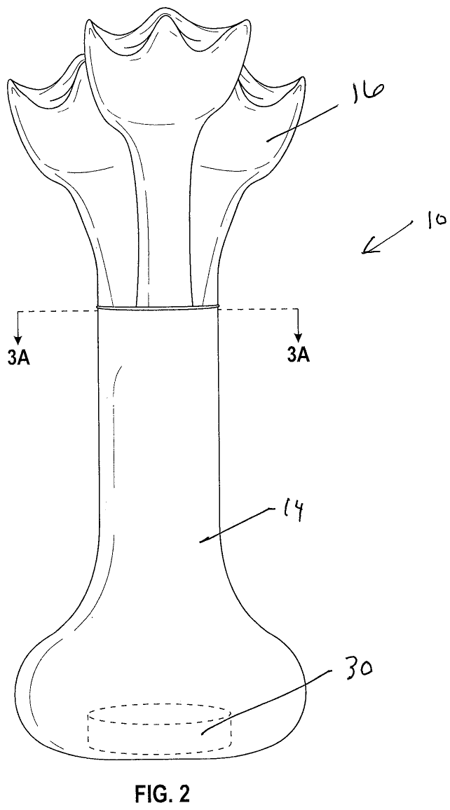

An inflatable and scented vase and flower bouquet according to a preferred embodiment of the present invention will now be described with reference to FIG. 1A to 3B of the accompanying drawings.

Shown in FIGS. 1A and 1B are perspective views of the inflatable and scented vase and flower bouquet 10 having a singular housing constructed of polyvinyl chloride, also known as PVC. The housing defines a singular interior space 12 that is airtight so that, when inflated, the housing expands to form a vase portion 14 and flower portion 16 adjacent and connected to the vase portion 14. In other words, the housing has a unitary construction but includes a shape configuration indicative of a vase with flowers extending from a top of the vase portion 14. Preferably, the housing is constructed of a material that is resilient or has an elasticity so that it can expand when inflated and can return to its original condition when deflated. To be clear, the vase portion 14 and flower portion 16 share the interior space 12 so that introduction of air (ambient air or helium) fills the interior space of both portions. It is understood that the ultimate shape configuration of the inflatable device is dependent on how the inflatable body is cut and constructed when deflated, i.e., at the point of manufacturing.

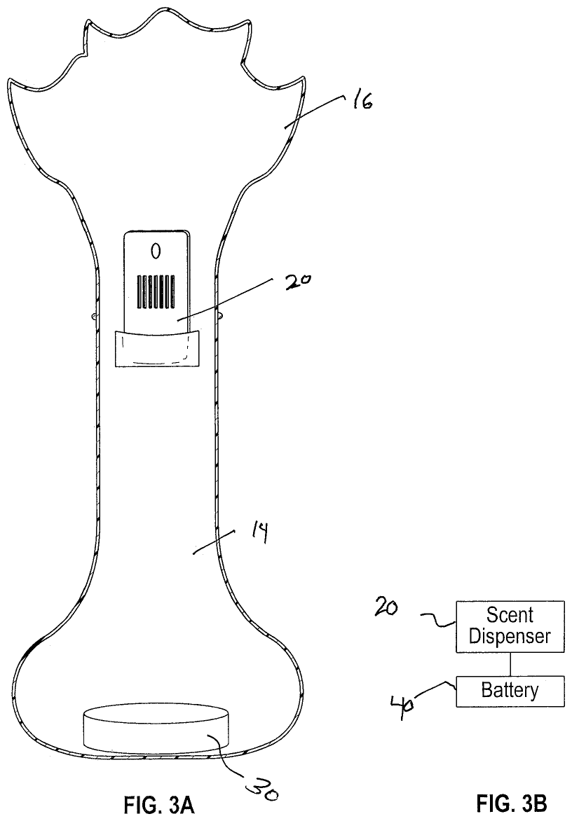

In another critical aspect, the inflatable and scented vase and flower bouquet 10 may include a scent dispenser 20 positioned in the interior space 12, such as being mounted to an inner surface of the housing proximate the flower portion 16. It is important that the scent dispenser 20 be located near the flower portion 16 or at least have an outlet vent positioned there so as to enhance a user's perception that the flowers are authentic. The scent dispenser may include a reservoir configured to hold a predetermined volume of liquid or powder scent associated with the type of flower depicted by the flower portion 16 of the housing. In an embodiment, the reservoir is not refillable as the inflatable and scented vase and flower bouquet 10 may be a single use product. In an embodiment, the scent dispenser 20 may have an activation button for manual actuation of the reservoir or may be situated on an exterior surface of the housing and have a scratch-and-sniff activation. In an embodiment, the scent dispenser 20 may be electrically coupled to a battery 40 and may be programmed at the factory (or have equivalent electronics) to emit the predetermined amount of scent, such as according to a timer, e.g., once every 3 hours (FIG. 3B).

In another embodiment, the inflatable and scented vase and flower bouquet 10 may include a weight 30 or ballast situated in the vase portion 14 of the interior space 12 (FIG. 3A). The weight is critical so that the interior space 12 of the housing may be inflated with helium and not float away as it may not be tethered as are traditional balloons.

Accordingly, the inflatable and scented vase and flower bouquet 10 enables a consumer to spontaneously purchase an inflatable flower and vase arrangement for a friend, family, or loved one without the disadvantages and planning of a “live” and traditional arrangement.

It is understood that while certain forms of this invention have been illustrated and described, it is not limited thereto except insofar as such limitations are included in the following claims and allowable functional equivalents thereof.

1. An artificial flower and vase device that is inflatable and scented, comprising:
a housing having a vase portion and a flower portion extending upwardly from the vase portion, said vase and flower portions including a continuous wall that defines an interior space into which gas is received so as to inflate said interior space and change said housing from a flattened and uninflated configuration to an expanded and inflated configuration indicative of a vase and flower arrangement;
wherein said housing is constructed of a material that is elastic and resilient and that is sealed against gas leakage;
a battery positioned in said interior space; and
a scent dispenser situated in said interior space and electrically connected to said battery, said scent dispenser having a reservoir for containing a liquid or powdered scent associated with a type of flower, said scent dispenser being operative to expel a portion of said scent when actuated.

2. The artificial flower and vase device as in claim 1, further comprising a ballast member situated in said interior space and having a predetermined weight.

3. The artificial flower and vase device as in claim 1, wherein said scent dispenser is attached to an inner surface of said housing adjacent said flower portion so as to enhance a user perception that the scent is associated with a flower of a type indicated by a configuration of the flower portion.

4. The artificial flower and vase device as in claim 1, wherein the gas is helium.

5. The artificial flower and vase device as in claim 1, wherein said continuous wall of said vase and flower portions of said housing includes a plurality of panels arranged in patterns indicative of an authentic vase and live flowers, respectively.

6. The artificial flower and vase device as in claim 1, wherein said vase portion of said housing includes a flat bottom, a lower section defining a diameter that is greater than a diameter defined by an upper section such that said housing stands upright when said housing is in said expanded and inflated configuration and said bottom is positioned on a level surface.

7. The artificial flower and vase device as in claim 1, wherein said scent dispenser is programmed to emit said predetermined portion of said scent according to a predetermined timing cycle.

8. The artificial flower and vase device as in claim 1, wherein said scent dispenser includes an activation button and is configured to emit said predetermined portion of said scent when said input button is manually actuated.

9. The artificial flower and vase device as in claim 1, wherein said scent dispenser is positioned on an exterior surface of said housing and includes a scratch-and-sniff construction having the scent associated with a type of flower.

10. The artificial flower and vase device as in claim 1, wherein said continuous wall of said housing is constructed of polyvinyl chloride.

11. An artificial flower and vase device that is inflatable and scented, comprising:
a housing having a vase portion and a flower portion extending upwardly from the vase portion, said vase and flower portions including a continuous wall that defines an interior space into which gas is received so as to inflate said interior space and change said housing from a flattened and uninflated configuration to an expanded and inflated configuration indicative of a vase and flower bouquet;
wherein said housing is constructed of polyvinyl chloride having a thickness that is flexible, elastic, and resilient so as to move between (1) said flattened and uninflated configuration and (2) said expanded and inflated configuration;
wherein said vase portion of said housing includes a flat bottom, a lower section defining a diameter that is greater than a diameter defined by an upper section such that said housing stands upright when said housing is in said expanded and inflated configuration and said bottom is positioned on a level surface;
a battery positioned in said interior space;
a scent dispenser situated in said interior space and electrically connected to said battery, said scent dispenser having a reservoir for containing a liquid or powdered scent associated with a type of flower, said scent dispenser being operative to expel a portion of said scent when actuated; and
a ballast member situated in said interior space and having a predetermined weight.

12. The artificial flower and vase device as in claim 11, wherein said scent dispenser is attached to an inner surface of said housing adjacent said flower portion so as to enhance a user perception that the scent is associated with a flower of a type indicated by a configuration of the flower portion.

13. The artificial flower and vase device as in claim 11, wherein the gas is helium.

14. The artificial flower and vase device as in claim 11, wherein said continuous wall of said vase and flower portions of said housing includes a plurality of panels arranged in patterns indicative of an authentic vase and live flowers, respectively.

15. The artificial flower and vase device as in claim 11, wherein said scent dispenser is programmed to emit said predetermined portion of said scent according to a predetermined timing cycle.

16. The artificial flower and vase device as in claim 11, wherein said scent dispenser includes an activation button and is configured to emit said predetermined portion of said scent when said input button is manually actuated.

17. The artificial flower and vase device as in claim 11, wherein said scent dispenser is positioned on an exterior surface of said housing and having a scratch-and-sniff construction that includes the scent associated with a type of flower.
